(19) United States
(12) Patent Application Publication
(10) Pub. No.: US 2003/0229269 A1
(43) Pub. Date: Dec. 11, 2003

(54) Scope sleeve

(76) Inventor: Robert N. Humphrey, Seattle, WA (US)

Correspondence Address: Lawrence D. Graham, Esq., BLACK LOWE & GRAHAM PLLC, 816 Second Avenue, Seattle, WA, 98104, US

(21) Appl. No.: 10/163,563

(22) Filed: Jun. 5, 2002

(51) Int. Cl. A61B 1/00

(52) U.S. Cl. 600/114; 600/121

(57) ABSTRACT

A scope cover is disclosed for use with a scope such as an endoscope that is inserted into a cavity. The scope cover preferably includes an elongated tubular body having internal ribs or grooves to retain a lubricant. An external flange extends radially outward from the tube body to prevent the tube from being inserted too far into the cavity. In use, the scope is inserted into the scope cover and both the scope and scope cover are inserted into the cavity. The scope cover is inserted no more than the depth of the flange, while the scope can continue to be inserted by sliding it through the scope cover.

### FIELD OF THE INVENTION

This invention relates generally to scope covering devices, including sleeves for covering boroscopes and endoscopes for a variety of uses.

### BACKGROUND OF THE INVENTION

Recent advances have produced miniature cameras that provide the ability to see inside remote or difficult to reach areas. One miniature camera tool is called an endoscope, typically defined as an instrument used to look inside the body. In most cases, an endoscope consists of a fiber-optic tube attached to a viewing device. The viewing device may take the form of an eyepiece, or may be electronically coupled to a computer system to display the captured images on a computer display. Endoscopes have a variety of uses, including exploration and biopsy of the colon, the bronchi of the lungs, and other areas. By adding miniature television cameras an incorporating surgical instruments, specialized endoscopes allow exploration and endoscopic surgery through small incisions.

A similar device used in industrial applications is typically called a borescope. As with endoscopes, borescopes come in a variety of shapes and sizes, and can be rigid or flexible. Borescopes are used, for example, to allow engineers or technicians to see inside an automotive or aircraft engine without dismantling the engine.

Endoscopes are particularly useful in performing a colonoscopy or sigmoidoscopy. A colonoscopy is an examination of the lining of the large intestine (also called the colon) by inserting an endoscope into the anus and through the colon. In some cases, abnormalities are first found by taking X-rays and then confirmed by the colonoscopy. Other abnormalities that are too small to be seen on an X-ray can also be detected in the colonoscopy. If a suspicious area is found, a doctor can perform a biopsy to examine the affected tissue.

For some time, rigid endoscopes were used to perform colonoscopies. Although they were useful, they only allowed viewing of a limited portion of the colon just inside the anus because the colon makes several sharp turns as it winds its way around the abdomen. The advent of flexible endoscopes now allows examination of more of the colon than was possible with rigid endoscopes.

Although new flexible endoscopes allow a more thorough colon examination, they suffer from a number of disadvantages. For example, as the endoscope is fed into the body, it slides along and presses against the inner walls of the colon. The pressure and sliding action can rupture the colon, particularly where the colon makes its first turn inside the anus just above the rectum.

In addition, endoscopes used in colon examinations are difficult to thoroughly clean between uses. Consequently, the abrasion of the scope against the side of the colon can potentially lead to infection, particularly if the endoscope is not sterile.

The use of borescopes in other settings also encounters problems. For example, when inserting a borescope into a small opening within an automobile engine, it is difficult to avoid a collision between the head of the scope and parts of the engine. This is particularly true when the scope must wind its way in and around various internal parts, or when it must pass through a small opening. If the head of the scope strikes against a metal object as it is inserted, the camera lens may break, requiring the scope to be replaced.

Consequently, there is a need for a protective scope cover that overcomes some of the above problems.

### SUMMARY OF THE INVENTION

The present invention comprises a cover for a scope, such as an endoscope or a borescope. In a preferred embodiment, the scope cover includes a flexible tube adapted to receive a flexible scope. The scope cover further includes a flange extending radially outward from the tube to prevent the tube from being inserted more than a preferred distance.

In accordance with further preferred aspects of the invention, the tube is divided into two sections, including a first section that is intended to be inserted with a scope and a second section intended to remain outside the body. The outside diameter of the first section is slightly smaller than that of the second section, forming a seat for the flange so that it will not slide off the tube.

In accordance with other preferred aspects of the invention, the tube is formed from rubber, latex, or other flexible materials.

In accordance with still further preferred aspects of the invention, the tube includes internal longitudinal grooves to assist in retaining a lubricant. The lubricant is applied to the inside of the tube or the outside of the scope before the scope is inserted into the tube.

In accordance with yet other preferred aspects of the invention, the tube may be constructed from more rigid materials, particularly for use in industrial applications.

In accordance with still another preferred aspect of the invention, the tube is fully removable from the scope. In surgical applications, this aspect makes the scope cover disposable and enhances sterility.

These and other preferred aspects are discussed in greater detail below.

### BRIEF DESCRIPTION OF THE DRAWINGS

The preferred and alternative embodiments of the present invention are described in detail below with reference to the following drawings.

FIG. 1 is a schematic view of a preferred scope cover shown in use with an endoscope and inserted into a colon;

FIG. 2 is a perspective view of a preferred scope cover;

FIG. 3 is a side view of a preferred scope cover; and

FIG. 4 is a front view of a preferred scope cover.

### DETAILED DESCRIPTION OF THE PREFERRED EMBODIMENT

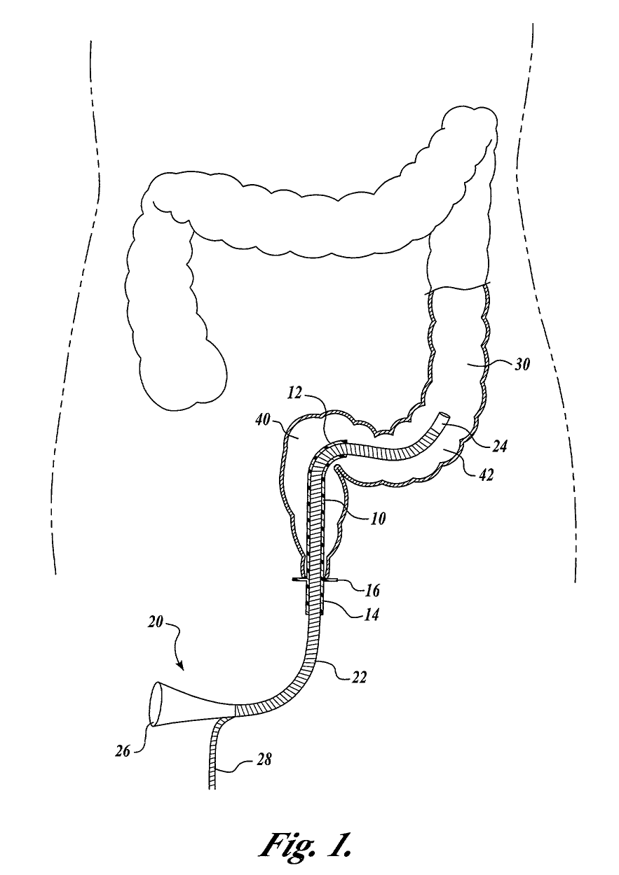

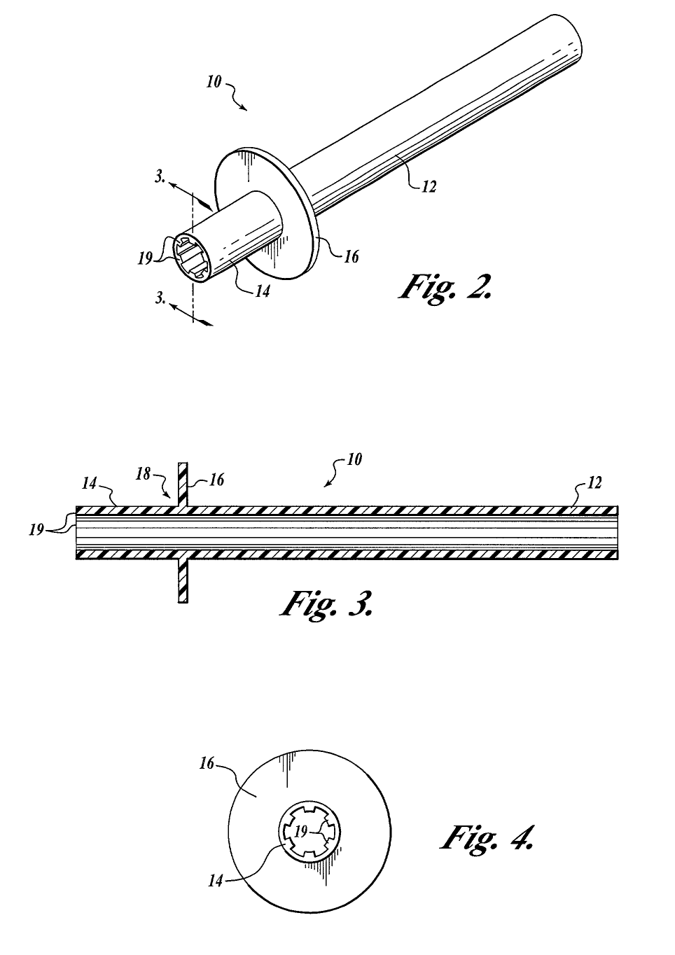

FIG. 1 illustrates a scope cover 10 in use with an endoscope 20 that has been inserted into a colon 30. The scope cover 10, also illustrated in FIGS. 2 and 3, includes a central tube having first section 12 and a second section 14 separated by a flange 16. The endoscope 20 comprises a flexible, substantially tubular scope body 22 with a camera lens 24 on the working end of the scope body 22 and configured to be inserted into the colon. Attached to the scope body 22 opposite the camera lens 24 is an eyepiece 26 through which a doctor or technician may view the colon walls during the examination. A light source 28 is also attached to the scope body 22 to provide light for the camera 24.

The tube of the scope cover 10 is preferably constructed from rubber, latex, or other flexible materials. A typical endoscope used in a colonoscopy has an outside diameter of between 2.8 and 3.2 mm. Accordingly, for use with an endoscope in a colonoscopy, the inside diameter of the scope tube is approximately 3.2 mm. Any inside diameter is possible, although the scope cover preferably includes an inside diameter that is within a few millimeters larger than the expected endoscope diameter.

The wall thickness of the first section 12 is about 0.25 mm, while the wall thickness of the second section 14 is about 1.5 mm. This difference in wall thicknesses produces a shoulder or seat 18 where the first section 12 meets the second section 14. The flange 16 is a circular disk having an outside diameter of about 40 mm and an opening at the center. The opening at the center of the flange 16 is approximately equal to the outside diameter of the first section 12 and smaller than the outside diameter of the second section 14. Consequently, the flange 16 can be produced separately and attached to the tube by sliding it along the first section 12 until it engages the seat 18.

The principal purpose of the different wall thicknesses and seat 18 is to provide a surface to engage and retain the flange 16. As an alternative, the tube may be formed from a uniform thickness but include either a raised area or a depressed area that will engage the flange 16. As another alternative, the tube and flange may be integrally formed, or the flange may be glued or otherwise permanently attached to the tube so that no seat or similar structure may be required.

The flange 16 is preferably formed of a rigid plastic to improve its ability to ensure that the second section 14 of the tube does not enter the anus or other body cavity. The tube may be larger or smaller, although 40 mm is preferred because it is large enough to prevent insertion but small enough to be easily handled.

The purpose of the flange 16 is principally to prevent insertion of the entire tube, including the second section 14. While a flange works well for this purpose, many alternatives are possible. For example, the flange may be replaced by a handle or extension that the technician may hold onto while inserting the scope. As another example, the second section may be sufficiently long that the technician is able to grasp the second section of the tube to prevent insertion. In such an embodiment, the tube need not have first and second sections with different wall thicknesses, but rather may be formed as one continuous tube with a uniform wall thickness. It may be preferable in such an embodiment to include one or more markings on the outside of the tube to allow the technician to see the extent to which the tube has been inserted. Still further, the flange need not be circular in shape, but rather may take any shape or size to allow it to serve as either a visual or physical barrier to insertion of the entire tube.

A clamp may also be used, instead of or in addition to the flange. If used, the clamp temporarily secures the scope cover to the scope during insertion of the scope, preferably at least through the first turn 40 of the lower portion of the colon 30. A first section 12 length of about 95 mm works well for this purpose, while a second section length of about 75 mm provides a sufficient area for the technician to manipulate the scope and cover. While these lengths are preferred, both the first and second sections may be longer or shorter. Once inserted approximately through the first turn 40, the clamp is removed, allowing the scope 20 to be further inserted while the scope cover 10 is held in place.

The inside walls of the scope cover include grooves or raised ridges to better retain a lubricant. A cross-sectional view of the scope tube is illustrated in FIG. 4, showing the flange 16 and second section 14. In the embodiment shown, a plurality of raised ridges 19 are provided on the inner wall. Alternatively, the inner wall may include grooves, rather than ridges. In either case, the grooves or ridges extend longitudinally over substantially the entire length of the scope tube.

When used with a colonoscopy, lubricant such as a lubricating jelly is applied to the interface between the scope and the scope cover. This can be accomplished in a variety of ways, such as placing lubricant inside the tube and then inserting the scope, or applying lubricant to the outside of the scope and then inserting it into the tube. Additional lubricant is preferably also applied to the outside of the scope cover.

Once lubricated, the scope and scope cover are inserted into the anus and into the colon. The scope cover assists the scope in making initial sharp turns inside the colon, preferably including at least the initial turn 40 but optionally also including a second sharp turn 42. After completing one or more of the sharp turns inside the anus so that the first section 12 is substantially fully inserted, the scope cover is held in place while the scope can be inserted further by sliding through the scope cover. In most cases, the scope will extend beyond the second end of the scope cover by an amount that exceeds the length of the second end of the scope cover. Thus, by sliding through the scope cover, the scope does not rub against the inside of the colon and the colon is protected against abrasion during insertion and removal.

After the colonoscopy is completed, the scope and scope cover are removed. The scope cover is intended to be disposable so that a new, sterile scope cover is used for each procedure. Accordingly, the scope cover improves cleanliness and reduces abrasion in the areas of greatest concern.

Although the scope cover is described above in use with a colonoscopy, it may also be used in other medical procedures. Thus, in any endoscopic procedure a scope cover can be incorporated to provide a tube through which an endoscope or other device may be passed to reduce pressure and abrasion.

Likewise, the scope cover may be used in non-medical settings. One such application is an automotive use in which a scope is inserted into an engine in order to inspect internal areas that otherwise could not be seen without dismantling the engine. In some cases, the scope must pass through small passages in order to reach an internal area to be inspected. If the scope camera lens 24 strikes against metal parts on its way into the inspection area, the lens may crack, essentially destroying the scope. In order to protect the scope against such damage, the scope is preferably inserted into the scope cover before inserting the scope into the engine. In this industrial embodiment, the scope cover 10 is preferably made from a rigid or semi-rigid material such as plastic. In an actual embodiment, the scope cover is has about the same size and rigidity as a standard drinking straw.

While the preferred embodiment of the invention has been illustrated and described, as noted above, many changes can be made without departing from the spirit and scope of the invention. Accordingly, the scope of the invention is not limited by the disclosure of the preferred embodiment.

The embodiments of the invention in which an exclusive property or privilege is claimed are defined as follows:

1. A scope cover for use with a scope of the type having a working end attached to a substantially tubular scope body, the scope cover comprising:
a substantially tubular scope cover body, the scope cover body having a first end and a second end, wherein the working end of the scope is removably insertable into the first end, through the scope body, and extendable out of the second end.

2. The scope cover of claim 1, wherein the scope cover body is formed from a flexible material.

3. The scope cover of claim 2, wherein the flexible material is latex.

4. The scope cover of claim 2, further comprising a means for preventing the second end of the scope body from being inserted into a cavity.

5. The scope cover of claim 2, further comprising a flange attached to the scope cover body between the first end and the second end.

6. The scope cover of claim 5, wherein the flange is substantially circular in cross-sectional shape.

7. The scope cover of claim 6, wherein the first end of the scope body and the second end of the scope body each have an outside diameter, and further wherein the outside diameter of the first end of the scope body is smaller than the outside diameter of the second end of the scope body to form a seat for engagement of the flange where the first end meets the second end.

8. The scope cover of claim 7, further comprising a plurality of grooves extending longitudinally on an inside surface of the scope body.

9. The scope cover of claim 7, further comprising a plurality of ridges extending longitudinally on an inside surface of the scope body.

10. The scope cover of claim 2, further comprising a marking on the scope body indicating a transition from the first end to the second end.

11. A method for inspecting an internal cavity with a scope of the type having a working end attached to a substantially tubular scope body, the method comprising:
placing the working end of the scope into a substantially tubular scope cover body, the scope cover body having a first end and a second end, wherein the working end of the scope is removably insertable into the first end, through the scope body, and extendable out of the second end; and
inserting the scope and scope cover into the cavity.

12. The method of claim 11, further comprising extending the working end out the second end of the scope cover.

13. The method of claim 12, wherein the scope cover body is formed from a flexible material.

14. The method of claim 13, wherein the flexible material is latex.

15. The method of claim 13, further comprising lubricating the scope or the scope cover or both.

16. The method of claim 15, wherein the scope is inserted into the cavity until the second end is approximately fully inserted.

17. The method of claim 16, wherein the second end of the scope cover is greater than about 80 mm in length.

18. The method of claim 17, wherein the working end of the scope is extended more than 80 mm beyond the second end of the scope cover.

19. A scope for accessing an internal cavity, comprising
a working end attached to a substantially tubular scope body; and
a scope cover having a substantially tubular scope cover body, the scope cover body having a first end and a second end, wherein the working end is removably insertable into the first end, through the scope body, and extendable out of the second end.

20. The scope of claim 19, wherein the working end is a camera lens.
